(19) United States
(12) Patent Application Publication
(10) Pub. No.: US 2021/0260355 A1
(43) Pub. Date: Aug. 26, 2021

(54) VALVED INTRODUCER SHEATH

(71) Applicant: NATIONAL UNIVERSITY OF IRELAND GALWAY, Galway (IE)

(72) Inventors: Jonathan BOUCHIER-HAYES, Dublin 6 (IE); Michael KELLY, Co. Clare (IE); Mark BRUZZI, Galway (IE)

(21) Appl. No.: 17/056,052

(22) PCT Filed: May 17, 2018
(86) PCT No.: PCT/EP2019/062546

§ 371 Date: Nov. 17, 2020

(30) Foreign Application Priority Data
May 17, 2018 (EP) 18172896.5

(51) Int. Cl. A61M 39/06; A61B 17/34

(52) U.S. Cl. CPC A61M 39/06; A61M 2039/0282; A61B 17/3423

(57) ABSTRACT

A valved introducer sheath (1) having a valve housing (17, 18) with a through-lumen and an inflatable cuff (2, 60, 70) disposed in the valve housing that can be inflated to occlude the through-lumen, is described. The device has an overflow tube (30) in fluidic connection with the inflatable cuff having a resiliently deformable overflow chamber (34). When the inflatable cuff is filled with inflation liquid, the cuff partly or fully occludes the through-lumen, thereby preventing blood pass through the lumen. When an interventional device is pushed through the lumen for use in a medical procedure, the pressure forces liquid from the cuff into the overflow tube, allowing the cuff to partly deflate. As the overflow chamber is resiliently deformable, the liquid is pressurised which maintains a seal between the inflatable cuff and the interventional device. Upon removal of the interventional device from the lumen, the overflow tube pushes fluid back into the cuff, allowing it to reinflate fully.

### FIELD OF THE INVENTION

The present invention relates to a valved introducer sheath.

### BACKGROUND TO THE INVENTION

Advances in technology and techniques have allowed clinicians to perform increasingly sophisticated procedures in a less invasive way. Endovascular approaches to procedures such as aneurysm repair and aortic valve repair have become the norm. Vascular access plays a key role in the safe and reliable delivery of these technologies into the bloodstream. The femoral artery remains the most commonly used access site for insertion of large-bore access sheaths and devices. The relatively large diameters of these devices pose a number of problems with respect to their introduction and to procedural workflow. There are a number of problems with current sheaths including blood loss through the haemostasis valve of the sheath and difficulty in pushing devices through the valve itself.

Valved Introducer Sheaths that can be used in diagnostic, therapeutic and interventional vascular procedures are commercially available, and are commonly used for introducing stents, vascular grafts, heart valves, occlusion devices or other implants and prostheses into a lumen or organ of a patient. Many haemostasis valves are composed of a resilient material such as a disk of silicone. This disk may contain a hole or a slit/s in the middle and the material will deform to allow the passage of a device through it while attempting to maintain a fluid tight seal. These types of disk valves are passive and can be problematic in that they create significant resistance when delivering a device through them. This can lead to damage of fragile devices through excessive force or kinking. The diameters of devices being passed through large bore introducer sheaths can range from a 0.035″ guidewire to greater than 0.25″/6 mm for some implants. This variation in diameters can mean an excessively tight seal may be required to accommodate all devices. This can also lead to the seal being damaged and haemostasis not being achieved.

An iris valve is a haemostasis valve that can be varied manually by rotating to seal on different diameter devices. This active valve overcomes some of the issues that can be encountered when using resilient passive disk valves in that different diameter devices can be passed through the valve without much force required. This reduces the operator effort and also the potential for damage to the device being passed. However, iris valves require regular adjustment to accommodate different device diameters while maintaining a fluid tight seal. During adjustment blood loss is often encountered. Also, iris valves are usually made of thin, flexible material to allow rotation from an open to closed position. This material is prone to damage when passing devices through it which leads to valve failure and blood loss.

Valved introducer sheaths are described in U.S. Pat. No. 5,871,474, WO99/24097, WO99/11308 and US2012/027116, all of which describe devices comprising a valve housing, a guide catheter, and an inflatable cuff.

It is an object of the invention to overcome at least one of the above-referenced problems.

### SUMMARY OF THE INVENTION

The present invention addresses the need for a valved introducer sheath that overcomes at least one problem of the prior art. This objective is met by providing a valved introducer sheath having a valve housing with a through-lumen and an inflatable cuff disposed in the valve housing that can be inflated to occlude the through-lumen. The device has an overflow tube in fluidic connection with the inflatable cuff having a resiliently deformable overflow chamber. When the inflatable cuff is filled with inflation liquid, the cuff partly or fully occludes the through-lumen, thereby preventing blood pass through the lumen. When an interventional device is pushed through the lumen for use in a medical procedure, the pressure forces liquid from the cuff into the overflow tube, allowing the cuff to partly deflate. As the overflow chamber is resiliently deformable, the liquid is pressurised which maintains a seal between the inflatable cuff and the interventional device. Upon removal of the interventional device from the lumen, the overflow tube pushes fluid back into the cuff, allowing it to reinflate fully. In addition, and in one embodiment, as the valve housing is provided in two interconnected parts and configured for rotational movement of a proximal part relative to a distal part, it is possible to twist the cuff with an interventional device in-situ tightening the cuff down onto the interventional device ensuring the best seal is achieved. This also enables the user to change between large and small diameter interventional devices without making any change to the volume of inflation system in the sheath by tweaking the amount of torsion exerted on the cuff. This enables a change between instruments while maintaining haemostasis. A second advantage of the sheath of the invention is that it enables one to relieve the seal tightness during motion of the instrument within the valve thus reducing the resistance during movement making the operability easier and reducing the risk of damage to the instrument during movement. This is not possible with the devices of the prior art, in which the proximal and distal ends are not configured for rotational adjustment. A third advantage is that increasing the tightness of the cuff axially clamps the interventional device in a stable position and inhibits any unwanted movement of the interventional device.

Thus, in one aspect, the invention provides valved introducer sheath assembly having a valve housing with a through-lumen, and an inflatable cuff disposed in the valve housing that can be inflated to fully or partially occlude the through-lumen, characterised in that the sheath assembly comprises an overflow tube in fluidic connection with the inflatable cuff having a resiliently deformable overflow chamber, whereby when an interventional device is pushed through the inflated inflatable cuff in the lumen for use in a medical procedure, pressure forces liquid from the cuff into the overflow tube, allowing the cuff to partly deflate while maintaining a seal between the inflatable cuff and the interventional device.

In one aspect, the valved introducer sheath assembly comprises:
a valve housing having a distal part, a proximal part configured for receipt of an interventional device, and a through lumen;
a guide catheter having a through lumen attached to the distal part of the valve housing; and
an inflatable cuff disposed within the through lumen of the valve housing configured for inflation during use to reduce the area of the through lumen and form a seal around an interventional device disposed within the inflatable cuff, the inflatable cuff having a distal end generally operatively connected to the distal part of the valve housing, a proximal end generally operatively connected to the proximal part of the valve housing, and an inflation port.

In one embodiment, the valved introducer sheath comprises an overflow tube comprising a resiliently deformable chamber configured for fluidic connection with the inflation port and transfer of inflation liquid between the inflation port and overflow tube, for example receipt of inflation liquid from the inflatable cuff when an interventional device is pushed through the introducer sheath, or transfer of inflation liquid to the inflatable cuff when an interventional device is removed from the introducer sheath. As the overflow chamber is resiliently deformable, the liquid is pressurised which maintains a seal between the inflatable cuff and the interventional device, and allows re-inflation of the inflatable cuff when the interventional device is retracted from the sheath.

In one embodiment, the valve housing comprises a distal part directly connected to a proximal part. In one embodiment, the inflatable cuff is disposed fully within the valve housing. In one embodiment, the valve housing is configured for rotational adjustment of the proximal part relative to the distal part to twist the inflatable cuff. Typically, the valve housing comprises locking means for locking the proximal part in a rotationally adjusted position with respect to the distal part. Various locking means can be provided the details of which will be apparent to a person skilled in the art, including a ratchet-type mechanism or a compression locking mechanism.

In one embodiment, the valved introducer sheath comprises a removable dilator configured for disposal within the through lumen of the valve housing and guide catheter. In one embodiment, the dilator has a through lumen configured for receipt of a guidewire. In one embodiment, the dilator is configured for locking attachment to the valve housing, typically a proximal end of the valve housing. The dilator is generally fully inserted into the sheath during placement of the device in the vasculature, and the inflatable cuff will be inflated to form a seal around the dilator, maintaining haemostasis during the insertion of the sheath. At this stage, a guidewire will be inserted through the lumen in the dilator. Upon removal of the dilator, leaving the guidewire in-situ in the guide catheter, the inflatable cuff will inflate due to the pressure exerted by the overflow tube and form a seal around the guidewire. An interventional device can then be threaded through the sheath whereby the inflatable cuff will form a seal around the device, maintaining haemostasis.

In one embodiment, the device is configured to form a seal around an interventional device threaded through the valve housing having a diameter of from 0.025″ to 0.5″, preferably from 0.035″ to 0.25″. In one embodiment, the device is configured to form a seal around a guidewire and a large-bore interventional device ranging from 12 Fr to 26 Fr, or larger.

Various forms of inflatable cuffs are envisaged, including cuffs from two tubes, one tube turned in on itself, and one tube and an inner wall of the valve housing. The tube or tubes generally are formed from a resiliently deformable material. In an embodiment in which two tubes are employed (i.e. an inner and an outer tube), the inner tube may have greater flexibility (more deformable) than the outer tube (i.e. it may be a different material, or it may be a thinner material). One or both end of the tube (or tubes) may comprise a seam (i.e. an annular region of increased thickness that is useful for forming a seal against another tube of part of the valve housing). In one embodiment, the seam comprises an annular sealing beading, like on O-ring. This may extend fully or partially around the tube. When two tubes are employed to form the inflatable cuff, they are generally sealed together at or close to their ends, for example by adhesive or welding (i.e. heat welding or ultrasound welding).

In one embodiment, the inflation cuff comprises an outer tube of resiliently deformable material and an inner tube of resiliently deformable material sealed together at each end thereof. This embodiment is illustrated in FIGS. 1 and 5. The inner tube may be tapered inwardly towards its centre (as shown in FIGS. 1 and 7). The advantage of this is that less deformation of the inner tube material is required during inflation to completely occlude the through lumen, thus reducing the risk of the valve leaking or being damaged when an interventional device is passed through the sheath. Another advantage related to tapering the inner elastomeric sleeve is that the excess material can be used to form seals with instruments which do not follow a perfectly circular shape or if two devices are being inserted through the valve at the same time the material has the ability to conform to the shape being inserted through. The angle and length of taper used can be varied depending on material elasticity, conformance requirements and other material properties. Another method to achieve similar results is to create the valve longer than the designed rigid housing, the extra material can also help to conform around the device being inserted there through.

In one embodiment, the distal part of the valve housing is configured to receive distal ends of the inner and outer tubes, whereby attachment of a guide catheter to the distal end of the valve housing clamps the distal ends of the inner and outer tubes together between the guide catheter and the distal part of the valve housing. For example, an inner wall of the distal part of the valve housing may comprise an annular slot configured to receive distal end of the inner and outer tubes, for example sealing seams or beading disposed at the ends of the inner and outer tubes. The annular slot may be dimensioned to receive the sealing seams or beads in a side-by-side disposition.

In one embodiment, the proximal part of the valve housing comprises a first part (for example an outer annular part) and a cooperating second part (for example an inner annular part), configured such that engagement of the first and second parts clamps the proximal end of the inner and outer tubes together between the outer and inner annular parts.

In one embodiment, the distal ends of the inner and outer tubes comprise sealing O-rings. In one embodiment, the proximal ends of the inner and outer tubes comprise sealing O-rings.

In one embodiment, the distal and proximal parts of the valve housing each comprise cooperating concentric sealing rings for sealing the ends of the inner and outer tubes together between the sealing rings.

In one embodiment, an inside wall of the inflation cuff adjacent the inflation port comprises spacer formations configured to prevent the cuff sealing during removal of an inflation liquid. The formations may comprise a series of ribs, or raised formations. In one embodiment, the spacer formations comprise a series of ribs that extend axially along the inflatable cuff adjacent the inflation port.

In one embodiment, the inflation port is disposed at a distal end of the inflation cuff, and in which the distal part of the valve housing comprises a channel having a proximal end configured to receive the inflation port and a distal end configured to receive a proximal end of the overflow tube. In one embodiment, the channel is substantially L-shaped.

In one embodiment, the overflow tube comprises one or more liquid outlets, and the resiliently deformable chamber comprises an inflatable balloon mounted on the overflow tube in fluidic connection with the one or more liquid outlets.

In one embodiment, the overflow tube comprises an inner tube and an outer tube that define an inflation lumen between the inner and outer tubes, and in which the resiliently deformable chamber comprises an inflatable balloon having a proximal end mounted on the outer tube and a distal end mounted on the inner tube.

Both of the above-referenced embodiments allow the use of an inflation tube that is more rigid that the resiliently deformable chamber, and therefore allow the inflation tube to be used to apply a vacuum to the inflation liquid in the inflation cuff to withdraw any air from the liquid.

In one embodiment, the inflatable cuff is defined by a tube of resiliently deformable material sealed at each end to an inner surface of the valve housing. This embodiment is illustrated in FIG. 4. In this embodiment, the distal and proximal parts of the valve housing may be interconnected by means of a rotation joint intermediate the ends of the valve housing. In one embodiment, the rotation joint comprises an O-ring seal.

In one embodiment, the inflatable cuff is defined by a tube of resiliently deformable material folded in (or out) on itself to provide an inner and outer tube that are sealed together at free ends thereof. This embodiment is illustrated in FIG. 6.

In the embodiments described herein, the inflatable cuff comprises one inflation port, and an overflow tube which is employed to also inflate the inflatable cuff. However, it will be appreciated that the inflatable cuff may comprise two or more fluidic ports, one for inflation and one for fluidic connection with the overflow tube. Likewise, the assembly may comprise one fluidic port and an overflow tube and a separate inflation tube, where the inflation tube is connected to the fluidic port for inflation of the inflatable cuff, and the inflation tube is then detached from the fluidic port and replaced with the overflow tube.

In another aspect, the invention provides a method of performing a transluminal interventional procedure on an individual that employs a valved introducer sheath according to the invention, the method comprising the steps of:
inserting a valved introducer sheath according to the invention into a blood vessel, in which the inflatable cuff is inflated to occlude the through lumen;
threading an interventional device through the through-lumen of the valve housing, whereby the inflatable cuff forms a seal around the interventional device.

In one embodiment, the valved introducer sheath when introduced into the blood vessel comprises a dilator disposed within the through lumen of the guide catheter and valve housing, and the inflatable cuff is inflated to form a seal around the dilator. In one embodiment, a guidewire for the interventional device is disposed within a through lumen of the dilator, wherein the method includes a step of retracting the dilator prior to insertion of the interventional device, leaving the guidewire in-situ in the blood vessel, whereby the inflatable cuff forms a seal around the guidewire,

In one embodiment, the process comprises a step of rotating the proximal part of the valve housing relative to the distal part of the valve housing to twist the inflatable cuff about a longitudinal axis and improve the seal between the inflatable cuff and the interventional device (or dilator), whereby the proximal part is maintained in the rotationally adjusted position.

In one embodiment, a second interventional device is inserted into the blood vessel though the valve housing, whereby the inflatable cuff deflates sufficiently to allow passage of the second interventional device through the valve housing while maintain partial or complete haemostasis.

In one embodiment, the first interventional device is a guidewire. In one embodiment, the second interventional device is a catheter device.

In one embodiment, the individual is a human.

In one embodiment, the blood vessel is a vein. In one embodiment, the blood vessel is an artery. In one embodiment, the artery is a femoral artery.

In one embodiment, the transluminal interventional procedure is a diagnostic procedure. In one embodiment, the transluminal interventional procedure is a therapeutic procedure. In one embodiment, the transluminal interventional procedure is a cardiac procedure (for example thrombolysis, coronary angioplasty, coronary artery bypass surgery, coronary stent placement).

Other aspects and preferred embodiments of the invention are defined and described in the other claims set out below.

### BRIEF DESCRIPTION OF THE FIGURES

FIG. 1 illustrates an inflatable cuff forming part of the valved introducer sheath of the invention, and formed from an inner and outer tubes.

FIG. 2 is a sectional side elevational view of the inflatable cuff of FIG. 1.

FIG. 3 is a sectional side elevational view of a valved introducer sheath of the invention (without the overflow tube), showing the inflatable cuff clamped in position in the through-lumen of the valve housing.

FIG. 4 is a sectional side elevational view of a valved introducer sheath of FIG. 3 and including the inflation liquid overflow tube.

FIG. 5 is a perspective, partly cut-away, view of a valved introducer sheath of FIG. 3.

FIG. 6 is a sectional view of a valved introducer sheath according to an alternative embodiment of the invention, in which the inflatable cuff is formed from a single tube and defined the single tube and an inner wall of the valve housing.

FIG. 7 is a sectional side elevational view of an alternative version of an inflatable cuff forming part of the valved introducer sheath according to the invention.

FIG. 8 is a sectional side elevational view of an alternative version of an inflatable cuff forming part of the valved introducer sheath according to the invention.

FIG. 9 is a sectional view of a mounting system for the inflatable cuff forming part of the valved introducer sheath according to the invention.

FIG. 10 is another sectional side elevational view of the mounting system and inflatable cuff of FIG. 9.

FIGS. 11 and 12 are illustrations of overflow tubes forming part of the valved introducer sheath according to the invention.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

As used herein, the term “valved introducer sheath” means a tubular sheath having a valve housing with a distal end and a proximal end configured for receipt of an interventional device, a through-lumen dimensioned for receipt of one or more interventional devices employed in endovascular surgical procedures, and a guide catheter attached to the distal end of the valve housing. The sheath comprises a valve configured to prevent blood passing out of the body through the sheath during use (i.e. maintain haemostasis).

The term “guide catheter” refers to an elongated tube that in use extends into the vasculature and that provides a conduit for endovascular delivery of an interventional device to a target location location in the vasculature. The valved introducer sheath generally also includes a dilator.

The term “dilator” as used herein refers to an elongated tube configured for removable nesting within the through lumen of the sheath, generally extending from a proximal end of the valve housing to a distal end of the guide catheter. The purpose of the dilator is to facilitate atraumatic entry and maintain haemostasis during insertion of the guide catheter and sheath into the vasculature. The inflatable cuff is primed by injection of liquid into the overflow tube, which pressurises the liquid and inflates the inflatable cuff which forms a tight seal around the dilator. The dilator generally includes a through lumen configured for receipt of a guidewire.

As used herein, the term “inflatable cuff” refers to an inflatable and radially expandable member, typically a balloon, formed from a resiliently deformable material and having a generally tubular shape with a through-lumen when deflated and a partially or fully occluded through-lumen when fully inflated. Typically, the inflatable cuff is formed from a resiliently deformable polymer material. Typically, the balloon is a compliant balloon. The cuff may be formed from concentric tubes that are hermetically sealed at their ends, or it may be formed from a single tube disposed within the valve housing and sealed to the valve housing at each of end of the tube. The cuff may also be formed from a single tube which is folded back on itself (inwardly or outwardly) and sealed at its free ends. The tube or tubes that make up the cuff is/are generally formed from a medical grade resiliently deformable elastomeric material, for example silicone, polyurethane, chronoprene or ePTFE. All are suitable due to their ability to be manufactured in thin wall form such that they have the ability to conform tightly to the interventional device. They are also suitable to hold high pressure without leakage. All of these materials are either naturally lubricous or can undergo secondary processes to control lubricity which will enable interventional devices to slide through or be held in position when under pressure. In one embodiment, the inner balloon has a central section that tapers or bulges inwardly. In one embodiment, the outer balloon has a central section that tapers outwardly. In one embodiment, the ? each tube comprises an annular seam at one or both ends, for example an annular beading seal.

As used herein, the term “overflow tube” refers to a resiliently deformable chamber fluidically connected to the inflatable cuff and configured for receipt of an inflation liquid for inflation or deflation of the inflatable cuff. Prior to use of the sheath, inflation liquid may be injected into the overflow tube, for pressurisation of the system, for example 1-5 ml of liquid. The liquid is generally saline. The pressurised liquid will cause inflation of the inflatable cuff to occlude the though lumen in the valve housing, or form a seal around a dilator disposed in the valve housing. Upon removal of the dilator, the pressure in the system causes the cuff to inflate further forming a seal around the guidewire disposed in the sheath. Upon insertion of an interventional device through the inflated cuff, the liquid in the cuff is forced into the overflow tube and into the resiliently deformable chamber, which expands to accommodate the liquid and exerts pressure on the liquid thereby inflating the cuff into sealing abutment with the interventional device. The overflow tube generally comprises a tube member which provides fluidic connection between the fluid inlet of the inflatable cuff and the resiliently deformable chamber. A connector may be provided between the tube and the inlet. The tube member may be sufficiently rigid to maintain its shape when a vacuum is applied through the tube—this is required prior to use of the device to purge system of any air. The resiliently deformable chamber may comprise a balloon mounted on the tube. For example, the tube may comprise one or more apertures, and the balloon may be mounted to the tube covering the apertures. The tube generally comprises an inflation aperture, for example a standard luer fitting for connection to a syringe for inflation with inflation fluid, or for fluidic connection to a pump for drawing a vacuum on the cuff. The tube or tubes that make up the balloon section of the overtube is/are generally formed from a medical grade resiliently deformable elastomeric material, for example silicone, polyurethane or chronoprene. Chronoprene may be used for example due to its resilience under pressure and its ability to return to its original shape. This will be beneficial for the application as it will ensure the fluid in the overflow balloon is pushed back into the valve/cuff when the interventional device is withdrawn thus ensuring haemostasis is maintained.

As used herein, the term “interventional device” refers to a device that is delivered to a site in the body by means of endovascular delivery. Examples include catheter guidewires and catheter devices employed for diagnostic or therapeutic applications, for example angioplasty catheters, ablation catheters, stent delivery catheters, valve delivery catheters, septal occluder catheters. The device may have a diameter ranging from 0.025″ to 0.50″, or may be a large bore device of 12 Fr to 26 Fr, or larger.

As used herein, the term “FR” or “French” as employed to define the diameter of an interventional device or catheter refers to the French catheter scale or “French units” (Fr). 1 “French” or “Fr” is equivalent to 0.33 mm=0.013″= 1/77″ of diameter. The size in French units is roughly equal to the circumference of the catheter in millimeters.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

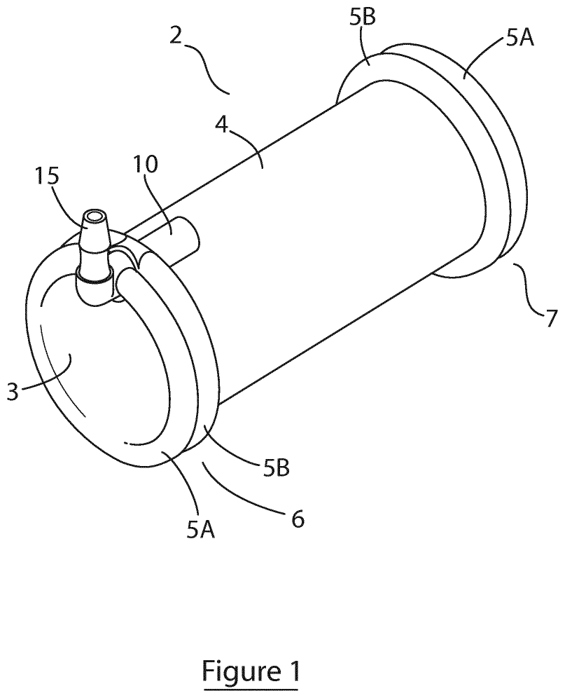

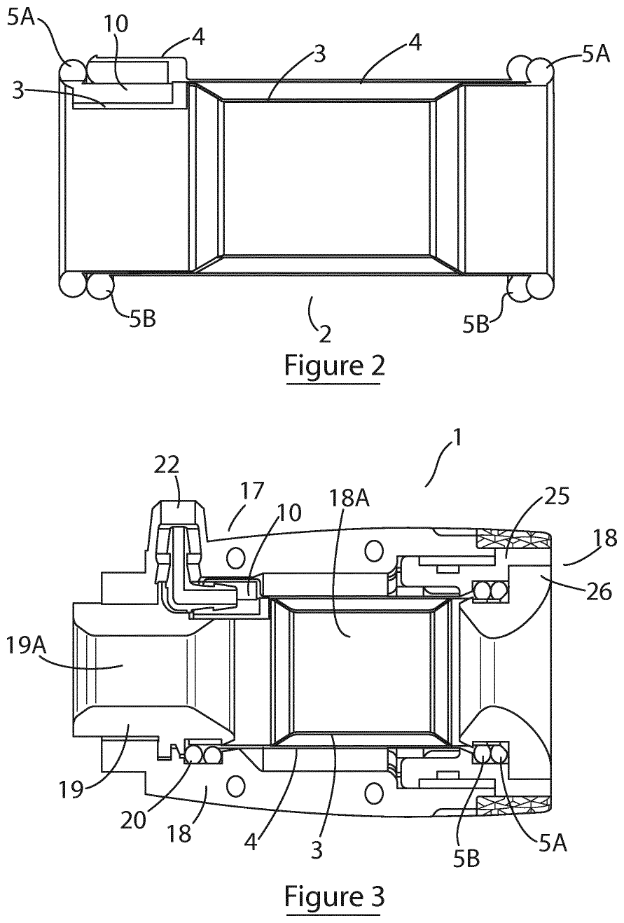

Referring initially to FIGS. 1 and 2, there is illustrated an inflatable cuff 2 forming part of a valved introducer sheath of the invention. The inflatable cuff 2 comprises an inner tube 3 formed of a medical grade resiliently deformable elastomer concentrically mounted within an outer tube 4 formed of a medical grade resiliently deformable elastomer. The inner tube 3 comprises annular sealing beading 5A at a distal end 6 and proximal end 7 of the cuff 2. Likewise, the outer tube 4 comprises sealing beading 5B at a distal end 8 and proximal end 9 thereof. The tubes are configured such that the beading 5A of the inner tube 3 and the beading 5B of the outer tube 4 are disposed in a side-by-side relationship when the inflatable cuff is assembled. As illustrated in FIGS. 1 and 2, the inner tube 3 and outer tube 4 are sealed together at each end, with the exception of an inflation port 10 disposed at a distal end of the cuff 2. The purpose of the inflation port is to receive an inflation tube during inflation of the device, and an overflow tube during use of the device. It will be appreciated that the overflow tube may be configured to perform both inflation and overflow functions. In FIG. 1, an L-shaped connector 15 is shown engaged with the inflation port.

Referring to FIGS. 3, 4 and 5, a valved introducer sheath according to the invention is shown, indicated generally by the reference numeral 1. The sheath 1 comprises a valve housing having a distal part 17, a cooperating proximal part 18 configured for engagement and relative rotational movement, and inflatable cuff 2 is disposed fully within the valve thus preventing the outer wall of the cuff expanding in an outward direction. This ensures that during inflation the cuff expands inwardly to close a through lumen in the valve housing.

The distal part 17 comprises a main body 18 having a through-lumen 18A and an end part 19 having a through lumen 19A that is co-extensive with the through-lumen 18A. The main body includes an annular slot 20 formed on an inner wall of the main body 18 dimensioned to receive the beading 5A, 5B of the inner tube 3 and outer tube 4, and an inflation tube aperture 22 configured to provide access to the inflation port 10 of the inflatable cuff 2. The main body 18 and end part 19 are configured to snap together clamping the beading 5A, 5B at the distal end of the inner and outer tubes together, and thereby securing the distal end of the inflatable cuff 2 to the distal part 17 of the valve housing. A guide catheter 23 is shown attached to the end part 19.

The proximal part 18 of the valve housing comprises two interconnecting parts, an outer annular part 25 having a through lumen and a cooperating inner annular part 26 having a through lumen that is coextensive with the through-lumen when the parts are connected. The outer annular part 25 is configured for mounting to an inside wall of the proximal end of the main body 18 for rotational movement thereon, and the inner annular part 26 is configured for snap-fitting to an inside wall of a proximal end of the outer annular part 25 clamping the proximal beading 5A, 5B together and securing the proximal end of the inflatable cuff 2 to the proximal part 18 of the valve housing. The fitting between the outer annular part 25 (distal part of valve housing) and main body 18 (proximal part of valve housing) is a compression fitting that allow rotational movement of the proximal part relative to the distal part, while locking the proximal part in a rotationally adjusted position.

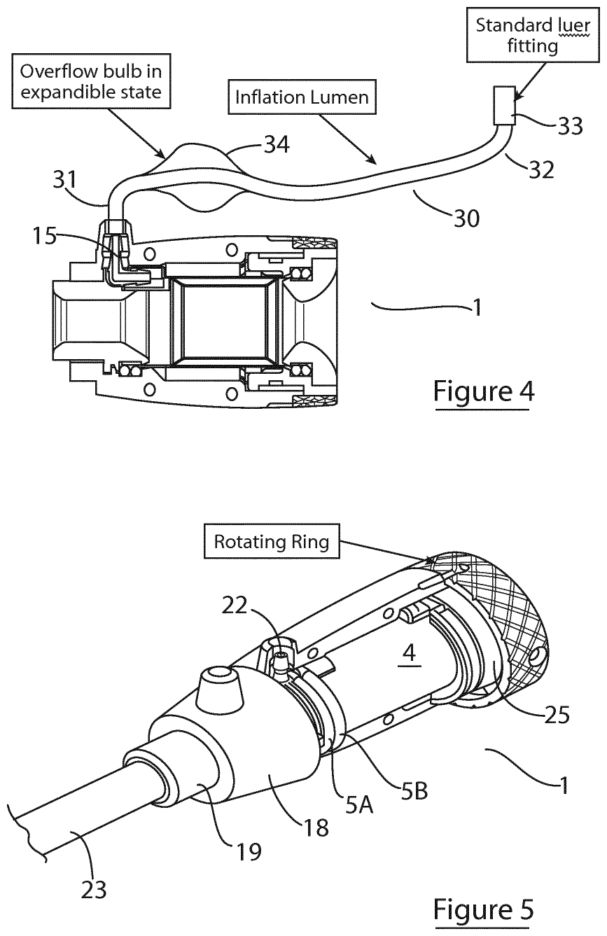

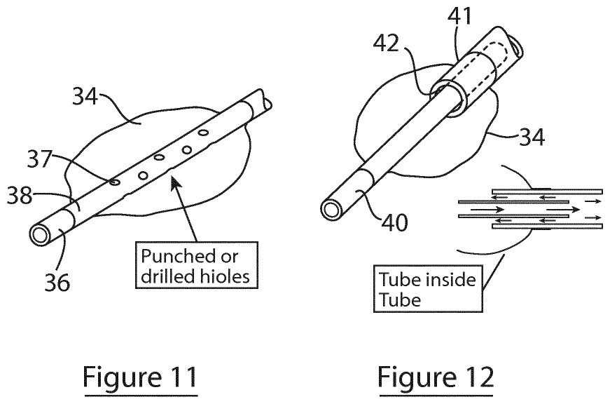

Referring specifically to FIG. 4, an overflow tube 30 is illustrated having a distal end 31 configured for connection to the L-shaped connector 15, a proximal end 32 comprising a standard luer fitting 33, and a resiliently deformable chamber, in this case an expandable balloon shown in a partly expanded state, 34 in fluidic connection with the inflation port for receipt of inflation liquid from the inflatable cuff when an interventional device is pushed through the introducer sheath. Referring to FIGS. 11 and 12, two specific embodiments of an overflow tube are described in which parts described with reference to the previous embodiments are assigned the same reference numerals. FIG. 11 shows an overflow tube 30 having an inner tube 36 including a series of apertures 37, and an outer tube 38 bearing a balloon 34 mounted to the inner tube over the apertures. The inner tube may be formed from a less deformable material then the outer tube and balloon, thereby allow a de-gassing vacuum to be drawn on the inner tube without the tube collapsing. FIG. 12 shows an alternative embodiment of the overflow tube, comprising an inner tube 40 (in fluidic connection with the inflatable cuff) and an outer tube 41 that define an inflation lumen 42 between the inner and outer tubes, and in which the inflatable balloon 34 has a proximal end mounted on the outer tube and a distal end mounted on the inner tube.

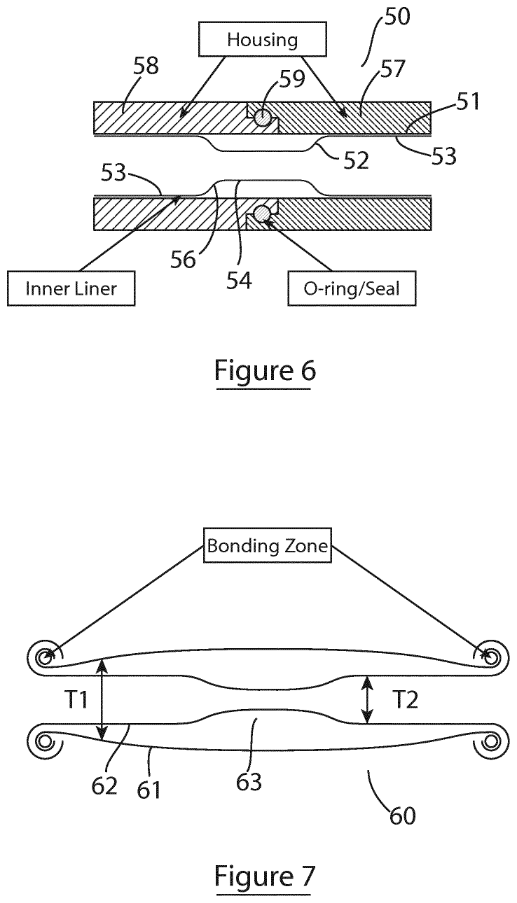

Referring to FIG. 6, there is illustrated an alternative embodiment of the valved introducer sheath according to the invention, indicated generally by the reference numerals 50, and in which parts described with reference to the previous embodiments are assigned the same reference numerals. In this embodiment, the inflatable cuff 2 is defined by a single tube 52 and an interior wall 51 of the two-part valve housing, the ends of the tube 51 being adhesively secured to the interior wall 52 at sealing section 53. The inner tube is designed such that the cuff has a central inflation section 54, peripheral sealing sections 53, and tapered sections 56 in between. The two-part housing comprises a proximal part 57 and distal part 58 configured for engagement and rotational movement of the proximal part relative to the distal part, and an O-ring 59 disposed in the rotational joint.

Referring to FIG. 7, there is illustrated an inflatable cuff forming part of an alternative embodiment of the valved introducer sheath according to the invention, and in which parts described with reference to the previous embodiments are assigned the same reference numerals. In this embodiment, the inflatable cuff 60 is defined by an outer tube 61 having a first diameter T1 mounted concentrically around an inner tube 62 having a second diameter T2. The outer tube 61 bulges outwardly slightly towards its centre, whereas the inner tube 62 bulges slightly inwardly towards its centre, and are sealed together at each end to provide the inflatable cuff having an inflatable central section 63.

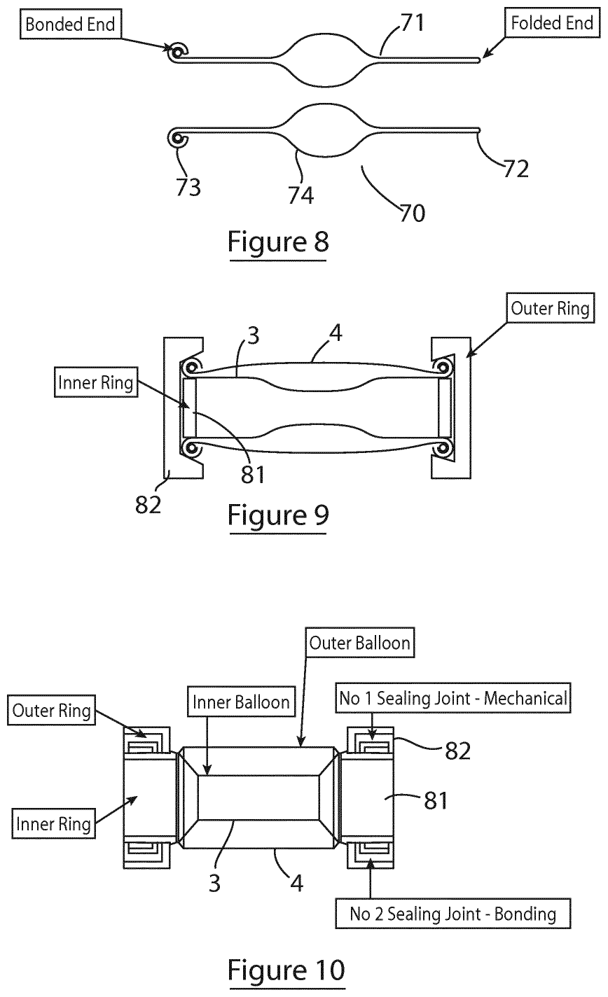

Referring to FIG. 8, there is illustrated an inflatable cuff forming part of an alternative embodiment of the valved introducer sheath according to the invention, and in which parts described with reference to the previous embodiments are assigned the same reference numerals. In this embodiment, the inflatable cuff 70 is defined by a single tube 71 that is folded in on its self to provide a folded end 72 and a bonded end 73, and an intermediate inflatable central section 74.

Referring to FIGS. 9 and 10, there is illustrated a part of a valve housing forming part of a valved introducer sheath according to an alternative embodiment of the invention, in which parts described with reference to the previous embodiments are assigned the same reference numerals. In this embodiment, the inflatable cuff is defined by inner and outer tubes 3, 4 as described previously, the ends of which are mounted between cooperating inner and outer concentric rings 81, 82 which are fitted together to clamp the end of the tube together. In addition, the abutting faces of the inner and outer tubes will be bonded together with a sealant to produce a double seal.

In one embodiment of the use of the sheath of the invention, the inflatable cuff is generally first purged of any air, by attaching a syringe to the overflow tube and applying a vacuum. Prior to or after the purging, the dilator is inserted into the through lumen of the sheath, extending from the proximal end of the valve housing to a distal end of the guide catheter. The proximal end of the dilator may be locked to the valve housing. The dilator generally includes a guidewire threaded through a through lumen in the dilator. The inflation liquid is then injected into the overflow tube, for example 2-3 ml, to pressurise the system, and inflate the cuff into sealing engagement with the dilator. The distal end of the guide catheter is then inserted into an opening in a blood vessel, and threaded into the blood vessel under imaging until the distal end is disposed at or close to a target location in the blood vessel. The dilator is then retracted proximally from the sheath, leaving the guidewire in-situ. When the distal end of the dilator is removed from the valve housing, the inflatable cuff expands to form a seal around the guidewire left in-situ, due to the pressure exerted on the liquid by the resiliently deformable chamber part of the overflow tube. The distal end of the dilator is generally tapered, which allows the cuff to gradually inflate. An interventional device is then threaded through the valve housing and guide catheter, along the guidewire, to a target location. As the distal end of the interventional device passes through the inflatable cuff, it forces the cuff to deflate forcing liquid into the overflow tube and resiliently deformable chamber, which expands to accommodate and further pressurise the liquid, increasing the inflation pressure on the cuff and improving the seal against the interventional device. If the seal is imperfect, and haemostasis is not achieved, the proximal end of the valve housing can be rotated to twist the cuff and tighten its engagement with the device, until haemostasis is achieved, whereupon the proximal end of the valve housing is maintained in the rotationally adjusted position until the procedure has been completed, whereby the interventional device and sheath are removed from the blood vessel, which is closed surgically.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A valved introducer sheath assembly (1) having a valve housing (17, 18) with a through-lumen, and an inflatable cuff (2, 60, 70) disposed in the valve housing that can be inflated to fully or partially occlude the through-lumen, characterised in that the sheath assembly comprises an overflow tube (30) in fluidic connection with the inflatable cuff having a resiliently deformable overflow chamber (34), whereby when an interventional device is pushed through the inflated inflatable cuff in the lumen for use in a medical procedure, pressure forces liquid from the cuff into the overflow tube, allowing the cuff to partly deflate while maintaining a seal between the inflatable cuff and the interventional device.

2. A valved introducer sheath assembly (1) according to claim 1, in which:
the valve housing has a distal part (17), a proximal part (18) configured for receipt of the interventional device;
a guide catheter (23) is operatively connected to the distal part of the guide housing;
the inflatable cuff has a distal end (6) operatively connected to the distal part (17) of the valve housing, a proximal end (7) operatively connected to the proximal part (18) of the valve housing, and an inflation port (10); and
the overflow tube (30) is configured for fluidic connection with the inflation port for inflating of the inflatable cuff and receipt of inflation liquid from the inflatable cuff when an interventional device is pushed through the introducer sheath.

3. A valved introducer sheath assembly according to claim 2, in which the valve housing is configured for rotational adjustment of the proximal part (18) relative to the distal part (17) to twist the inflatable cuff (2), wherein the valve housing comprises locking means for locking the proximal part in a rotationally adjusted position.

4. A valved introducer sheath assembly according to claim 1, in which the inflatable cuff when inflated comprises a central section (54) that blocks the through lumen, peripheral sections that do not block the through lumen (53), and tapered sections (52) in between.

5. A valved introducer sheath assembly according to claim 1, in which the inflation cuff comprises an outer tube (4) of resiliently deformable material and an inner tube (3) of resiliently deformable material sealed together at each end thereof.

6. A valved introducer sheath assembly according to claim 1, in which the inflation cuff comprises an outer tube (4) of resiliently deformable material and an inner tube (3) of resiliently deformable material sealed together at each end thereof, and in which the distal part (17) of the valve housing comprises cooperating parts (18, 19) configured for engagement to clamp the distal ends of the inner and outer tubes together between the parts.

7. A valved introducer sheath assembly according to claim 1, in which the inflation cuff comprises an outer tube (4) of resiliently deformable material and an inner tube (3) of resiliently deformable material sealed together at each end thereof, and in which the proximal part (18) of the valve housing comprises an outer annular part and a cooperating inner annular part, whereby attachment of the outer annular part to the inner annular part clamps the proximal end of the inner and outer tubes together between the outer and inner annular parts.

8. A valved introducer sheath assembly according to claim 1, in which the inflation cuff comprises an outer tube (4) of resiliently deformable material and an inner tube (3) of resiliently deformable material sealed together at each end thereof, and in which the distal ends of the inner and outer tubes comprise sealing O-rings (5A, 5B), and in which the distal part (17) of the valve housing optionally comprises an annular channel (20) configured to receive the sealing O-rings (5A, 5B) of the inner and outer tubes.

9. A valved introducer sheath assembly according to claim 1, in which the inflation cuff comprises an outer tube (4) of resiliently deformable material and an inner tube (3) of resiliently deformable material sealed together at each end thereof, and in which the proximal ends of the inner and outer tubes comprise sealing O-rings (5A, 5B).

10. A valved introducer sheath assembly according to claim 5, in which the inflation cuff comprises an outer tube (4) of resiliently deformable material and an inner tube (3) of resiliently deformable material sealed together at each end thereof, in which the distal and proximal parts (17, 18) of the valve housing each comprise cooperating concentric sealing rings (81, 82) for sealing the ends of the inner and outer tubes together.

11. A valved introducer sheath assembly according to claim 1, in which the overflow tube (30) comprises one or more liquid outlets (37), and the resiliently deformable chamber comprises an inflatable balloon (34) mounted on the overflow tube in fluidic connection with the one or more liquid outlets.

12. A valved introducer sheath assembly according to claim 1, in which the overflow tube (30) comprises an inner tube (4) and an outer tube (41) that define an inflation lumen (42) between the inner and outer tubes, and in which the resiliently deformable chamber comprises an inflatable balloon (34) having a proximal end mounted on the outer tube and a distal end mounted on the inner tube.

13. A valved introducer sheath assembly according to claim 1, in which the inflatable cuff is defined by a tube of resiliently deformable material (51) sealed at each end to an inner surface (51) of the valve housing.

14. A valved introducer sheath assembly according to claim 1, in which the inflatable cuff is defined by a tube of resiliently deformable material (51) sealed at each end to an inner surface (51) of the valve housing, and in which the distal and proximal parts (57, 58) of the valve housing are interconnected by means of a rotation joint intermediate the ends of the valve housing, in which the rotation joint optionally comprises an O-ring seal (59).

15. A valved introducer sheath assembly according to claim 1, in which the inflatable cuff (70) is defined by a tube of resiliently deformable material (71) folded in on itself to provide an inner and outer tube that are sealed together at free ends thereof.

16. A valved introducer sheath assembly according to claim 1, including a dilator configured for removable nesting within a through lumen of the sheath, extending from a proximal end of the valve housing to a distal end of the guide catheter, wherein the the dilator is configured for locking attachment to a proximal end of the valve housing.
